(19) United States
(12) Patent Application Publication
(10) Pub. No.: US 2006/0287560 A1
(43) Pub. Date: Dec. 21, 2006

(54) Process and a device for producing gasoline, kerosene and diesel oil from waste plastic, rubber and machine oil

(76) Inventor: Fusheng Xie, Zibo (CN)

Correspondence Address: ANNA WU, P.O. BOX 93286, CITY OF INDUSTRY, CA, 91715, US

(21) Appl. No.: 10/562,370

(22) PCT Filed: Jun. 22, 2004
(86) PCT No.: PCT/CN04/00663

§ 371 Date: Jul. 5, 2006

(30) Foreign Application Priority Data
Jun. 24, 2003 (CN) 03146751.2

(51) Int. Cl. C10G 1/10; C07C 4/00; C07C 1/00; C10G 1/00

(52) U.S. Cl. 585/241

(57) ABSTRACT

A process and a device that produce gasoline, kerosene and diesel oil from waste plastic, rubber and machine oil, the device comprises sussively cracker, fixed bed, packed tower, fractionating tower, the top part of the fractionating tower connects with gasoline quencher, and the quencher links condenser, separator of oil and water, filter, and gasoline treating column in series, the latter is connected with filter, finished product tank; the kerosene fraction from the middle part of the fractionating tower and the diesel oil fraction from the lower part enter condenser, separator of oil and water, filter, and treating column in series respectively, the residue from the bottom returns back to the cracker. In this invention, quartz and sand are added during cracking; one step process is used in fixed bed; a chemical agent for removing the odor and changing the color is added during the fractionation. The device in this invention has low cost and reduced volume, which simplifies the process and shorterns the production cycle, the oil product obtained has good quality and high yiel.

### FIELD OF TECHNOLOGY

The invention is related to a process and a device for producing gasoline, kerosene and diesel oil from waste plastic, rubber and machine oil.

### BACKGROUND OF TECHNOLOGY

There are a lot of methods to epurate gasoline, kerosene and diesel oil from waste plastics, so as to enable them recycled and valuable. The processes are, in general, that waste plastics are cracked catalytically and then components are fractionated. But all these processes have such disadvantages of unsatisfied quality of oil product and long cycle of production that influence the popularization and utilization of technology.

### DISCLOSURE OF INVENTION

The purpose of this invention is to provide a process and a device for producing gasoline, kerosene and diesel oil from waste plastic, rubber and machine oil, with a high quality of oil product and a short cycle of production.

The process of the invention for producing gasoline, kerosene and diesel oil from waste plastic, rubber and machine oil is characterized in:

(1) Adding quartz and sand into the waste raw materials, including waste plastics or materials containing waste plastic, waste rubbers and machine oil, then catalytically cracking the mixture at a temperiture of 50-480□;

(2) Further catalytically cracking the gas fraction obtained in step (1) in a fixed bed to obtain an oil stream;

(3) Fractionating the oil stream and collecting the fractions of gasoline, kerosene and diesel oil, respectively;

(4) Treating fractions of gasoline, kerosene and diesel oil, respectively.

In step (1), the amount of quartz is 0.3-1.5% of that of the cracking feed, and the amount of sand is 0.1-0.5% of that of the cracking feed. The catalytic cracking is proceeded in a cracker. During the process of cracking, the quartz and sand move in parallel at about 100□, and in curve when the temperature reaches 150□, then in beeline up and down when the temperature reaches 250□. The effects of such moving types are: 1. accelerating the cracking; 2. expanding bores in raw materials to get through channels; 3. improving the quality of the oil product by eliminating iron from the feed; 4. reducing coking in the cracker; 5. increasing the yield of oil products; and 6. shortening the production cycle. In the above effects, the elimination of iron is especially beneficial and has not been solved in the prior art. The existence of iron damages equipments and influences the quality of oil product since it is easy to combine with chlorin ion to form impurities. In this invention, one of the two sources of raw materials for cracking is the direct use of waste plastics (except PVC plastic) and waste rubbers; and the other is the use of waste plastics as main raw materials, including all formula ingredients containing waste plastics disclosed in the prior art. The catalyzers in the cracking can be those disclosed in the prior art. The cracking is a process with a temperature increased gradually, and gas fractions are collected immediately when they are produced. The proper cracking temperature is 60-460° C. for waste plastics, 80-480° C. for waste rubbers and 50-380° C. for waste machine oil, respectively.

The gases formed in the cracker are further catalytically cracked and adsorbed in a fixed bed. Two fixed beds were adopted and procedures were in two steps, in general, in the prior art. However, an one-step process is adopted in the present invention i.e., there is only one fixed bed in the process. In the fixed bed, the cracked gas is gotten rid of impurities through a coke layer with a thickness of 20-50 cm, then successively passes through a Pall ring layer, a sorbent layer and a catalyzer layer with 5 Å molecular sieves as the catalyzer. In this invention, gases first pass a coke layer to eliminate impurities so that one fixed bed is enough. The processes of catalytic cracking and adsorbing in the fixed bed are based on those in the prior art.

The gases obtained from the fixed bed are preferrably filtrated again in a packed tower to eliminate impurities and then be fractionated in a fractionating tower. The fraction from the top part of the fractionating tower, where the temperature is 195-198° C., is the gasoline fraction; that from the middle part, 200-230° C., is the kerosene fraction; and that from the bottom part, 300-360° C., is the diesel oil fraction. In order to eliminate the undesirable odor and improve the transparency of the oil product, following reagents are added into the fractionating tower every 5-8 hours during the fractionating process: 200 ppm of water solution of cobaltic phthalocyanin sulfonate just obtained by dissolving cobaltic phthalocyanin sulfonate in water; 1.5 kg of 10% NaOH; 3% o of 10% H₂O₂ calculated on the basis of the volume of 10% NaOH.

The gasoline fraction is condensed at a temperature of 160-180° C. The liquid fraction returns back to the fractionating tower and the gas fraction is condensed again until the temperature reduces to 30-60° C., followed by a sedimentation to separate the oil and water, and then the oil is filtered. At last, the gasoline is treated at a temperature of 30-50° C. In the treating process, active kaolin is added in an amount of 1-5% based on the weight of gasoline, i.e., the amount of actove kaolin added for producing 1 ton gasoline is 0.01-0.05 ton. The actions of active kaolin are eliminating impurities, removing the color of gasoline, improving the transparency and increasing the octane number.

The diesel oil fraction is condensed, sedimented and filtered successively, and finally treated. During the treating process, 98% H₂SO₄ is added in an amount of 2-5% of the weight of the diesel oil fraction to proceed an acid washing, then 96% NaOH is added in an amount of 1-3% of the weight of the diesel oil fraction to proceed an alkali washing, and finally a cetane additive is added in an amount of 1-5% of the weight of the diesel oil fraction so as to raise the cetane number of the diesel oil.

The kerosene fraction is condensed, sedimented and filtered successively, and finally treated in a treating tower in a general way.

A complete set of production device for the above process comprises in turn a cracker, a fixed bed, a packed tower, a fractionating tower, the top part of the fractionating tower connects with a gasoline quencher via a tube, and the quencher connects to a condenser, the condenser links to a separator of oil and water, a filter and a gasoline treating column in series, the gasoline treating column is connected with a filter, and a product tank in series; the middle part of the fractionating tower connects with a condenser, a separator of oil and water, a filter and a kerosene treating column in series, and the kerosene treating column connects a filter and a product tank in series; and the low part of the fractionating tower connects with a condenser, a separator of oil and water, a filter, and a diesel oil treating column in series, the diesel oil treating column connects to a filter and a product tank in series; the bottom of the fractionating tower connects to the cracker via a tube.

Said quencher which serves for condensing the gasoline fraction is in the shape of a pot, and comprises a water inlet tube, a water outlet tube, a cooling pipe, an oil pipe on the top of said quencher which is linked to a condenser, an “U” circumfluence pipe in the bottom of said quencher which is linked to the fractionating tower.

The virtues of the invention are as follows:

There are several equipments for eliminating impurities in the whole process so as to ensure the product quality. The investment for equipments is low and the bulks of equipments are reduced. The procedures are simplified and the production cycle is shortened. The oil products are of good quality and high transparency, looking like table-water. The gasoline obtained is a lead-free gasoline, with a low level of sulphur, reaching the National Standard of 93# gasoline. The finished products of oil can be obtained in a high yield. The yield is 65% of the amount of waste plastics, or 80-83% of the amount of the waste oil. The circumstance is free from pollution in the process of the invention, Waste plastics and rubbers are directly fed into the cracker without washing, whereas a large amount of water is needed for washing in the prior art. Liquefied gas produced during the process can be utilized as an energy source. The invention is especially applicable to the treatment of waste plastics.

### BRIEF DESCRIPTION OF DRAWINGS

FIG. 1 is a schematic diagram of the process and device of the invention.

FIG. 2 is a schematic diagram of the quencher, wherein:

1. cooling pipe; 2. flange; 3. valves for inlet and outlet of water; 4. water outlet tube; 5. water inlet tube; 6. outlet tube for light gasoline; 7. “U” circumfluence pipe; and 8. flange.

### PREFERRED EMBODIMENTS

The invention is exemplified, but not limited, by the following example in which waste plastics were utilized.

The waste plastics were fed into the cracker and cracked catalytically in it. The catalyst was formed by adsorbing active ZnCl to a carrier of particulate Al₂O₃ in virtue of dipping. The temperature in the cracker was increased gradually to 460° C., and the collection of gases began from 60° C. Quartz and sand were added, together with raw materials, into the cracker. The amount of quartz is 1% of the weight of raw materials being cracked, and the amout of sand is 0.2% of the weight of raw materials being cracked. The gases obtained from cracking were introduced into the fixed bed, which comprises, from the bottom to the top part, a coke layer in a thickness of 20-50 cm, a Pall ring layer, a sorbent layer containing lime as the sorbent in a thickness of 60-100 cm, and a catalyst layer containing 5 Å molecular creives in a thickness of 80-120 cm. The gases obtained from the fixed bed were introduced into the packed tower and filtered again there to adsorb impurities, and then introduced into the fractionating tower. The fraction from the top part of the fractionating tower, where the temperature was 195-198° C., was a gasoline fraction; the fraction from the middle part, 200-230° C., was a kerosene fraction; and the fraction from the bottom part, 300-360° C., was a diesel oil fraction. The following reagents were added into the fractionating tower from the top every 5-8 hours: 200 ppm of water solution of cobaltic phthalocyanin sulfonate which was just formed from dissolving cobaltic phthalocyanin sulfonate in water, 1.5 kg of 10% NaOH; 3% o 10% H₂O₂ calculated on the basis of the volume of 10% NaOH. The heavy oil coming from the bottom of the fractionating tower was returned to the cracker.

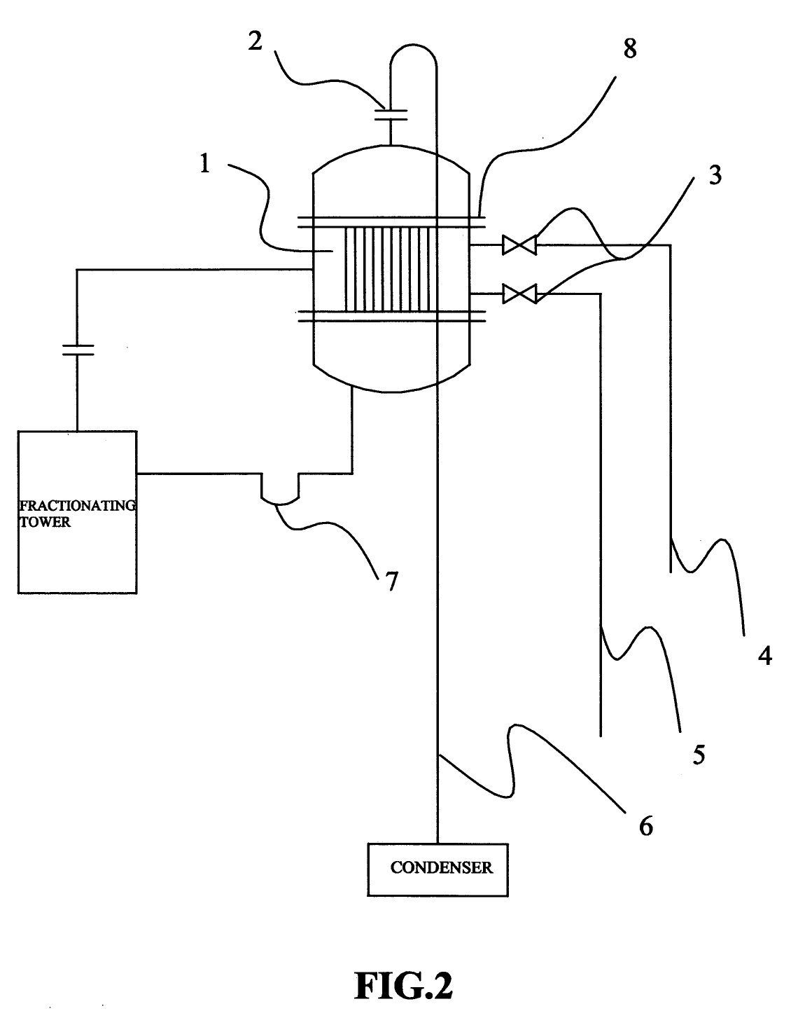

The gasoline fraction was cooled in a quencher (FIG. 2) with tap water. When the temperature reduced to 160-180° C., the liquid fraction obtained was returned back to the fractionating tower, through the “U,” shape circumfluence pipe, to be fractionated again, and the gas fraction, via outlet tube for light gasoline 6, was introduced into a condenser to be condensed until the temperature reduced to 30-60° C., then introduced into a separator of oil and water to have oil and water separated by sedimentation. The oil was introduced into a filter to be filtered again, then treated in a treating column at a temperature of 30-50° C., in the existance of active kaolin in an amount of 0.01-0.05 ton for producing 1 ton gasoline. The treated gasoline was introduced into a product tank after being filtered.

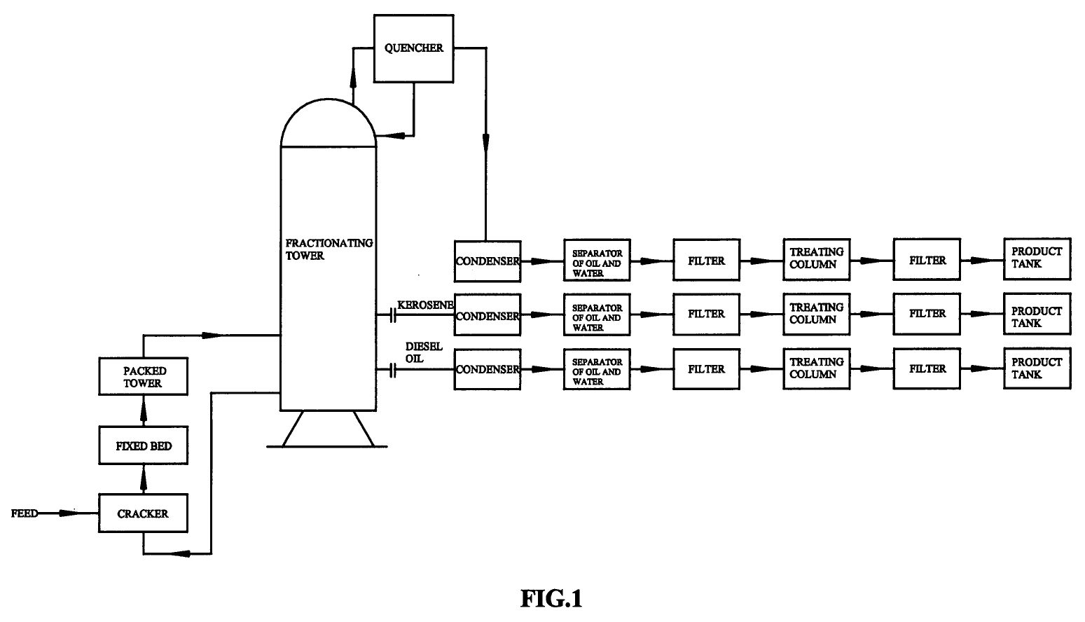

The kerosene fraction was condensed, sedimented, filtered and, finally treated in a treating tower. As shown in FIG. 1, the kerosene fraction entered directly into the condenser, without need for a quencher.

The diesel oil fraction was also condensed, sedimented, filtered and, finally treated. During the treating process, 98% H₂SO₄ is added in an amount of 2-5% of the weight of the diesel oil fraction to proceed an acid washing, then 96% NaOH is added in an amount of 1-3% of the weight of the diesel oil fraction to proceed an alkali washing, and finally a cetane additive is added in an amount of 1-5% of the weight of the diesel oil fraction.

As shown in FIG. 2, the quencher comprises water inlet tube 5, water outlet tube 4, two flanges 8, cooling pipe 1 between the two flangess, outlet tube for light gasoline 16 on the top of said quencher which is linked to a condenser, an “U” circumfluence pipe 7 in the bottom of said quencher which is linked to the fractionating tower.

The device and the process for producing gasoline, diesel oil and kerosene from waste or old rubbers and waste machine oil is the same as the above.

What is claimed is:

1. A process for producing gasoline, kerosene and diesel oil from waste plastic, rubber or machine oil, characterized in:
(1) Adding quartz and sand into the waste raw materials, including waste plastics or materials containing waste plastic, waste rubbers and machine oil, then catalytically cracking the mixture at a cracking temperature of 50-480□;
(2) Further catalytically cracking the gas fraction obtained in step (1) in a fixed bed to obtain an oil stream;
(3) Fractionating the oil stream and collecting the fractions of gasoline, kerosene and diesel oil, respectively;
(4) Treating fractions of gasoline, kerosene and diesel oil, respectively.

2. The process according to claim 1, wherein, in step (1), said quartz is added in an amount of 0.3-1.5% based on the weight of the cracking feed, and said sand is added in an amount of 0.1-0.5% based on the weight of the cracking feed.

3. The process according to claim 1, wherein, said cracking temperature in step (1) is gradually increased.

4. The process according to claim 1, wherein, in the fixed bed in step (2), the cracked gas is gotten rid of impurities through a coke layer in a thickness of 20-50 cm, then successively passes through a Pall ring layer, a sorbent layer and a catalyzer layer with 5 Å molecular sieves as the catalyzer.

5. The process according to claim 1, wherein, during the fractionating procedure in step (3), a mixture of cobaltic phthalocyanin sulfonate, NaOH and H₂O₂ is added every 5-8 hours, said mixture cotaining: 200 ppm of water solution of cobaltic phthalocyanin sulfonate just obtained by dissolving cobaltic phthalocyanin sulfonate in water; 1.5 kg of 10% NaOH; 3% of 10% H₂O₂ calculated on the basis of the volume of 10% NaOH.

6. The process according to claim 1, wherein, in step (3), the fraction from the top part of the fractionating tower, where the temperature is 195-198° C., is a gasoline fraction; the fraction from the middle part, 200-230° C., is a kerosene fraction; and the fraction from the bottom part, 300-360° C., is a diesel oil fraction.

7. The process according to claim 1 or claim 6, wherein, the gasoline fraction is condensed at a temperature of 160-180° C.; the liquid fraction returns back to the fractionating tower and the gas fraction is condensed again until the temperature reduces to 30-60° C., followed by a sedimentation to separate the oil and water, and the oil is filtered, then treated at a temperature of 30-50° C. in the existance of active kaolin in an amount of 1-5% based on the weight of gasoline

8. The process according to claim 1 or claim 6, wherein, the diesel oil fraction is treated, during said treating process, 98% H₂SO₄ is added in an amount of 2-5% based on the weight of the diesel oil fraction to proceed an acid washing, then 96% NaOH is added in an amount of 1-3% based on the weight of the diesel oil fraction to proceed an alkali washing, and finally a cetane additive is added in an amount of 1-5% based on the weight of the diesel oil fraction.

9. The process according to claim 1, wherein, waste plastics or materials containing waste plastics are used for producing gasoline, kerosene and diesel oil, and said cracking in step (1) is at a temperature of 60-460° C.

10. The process according to claim 1, wherein, waste rubbers are used for producing gasoline, kerosene and diesel oil, and said cracking in step (1) is at a temperature of 80-480′.

11. The process according to claim 1, wherein, waste machine oil is used for producing gasoline, kerosene and diesel oil, and said cracking in step (1) is at a temperature of 50-380° C.

12. A device for producing gasoline, kerosene and diesel oil from waste plastic, rubber or machine oil, comprising in turn a cracker, a fixed bed, a packed tower, a fractionating tower, the top part of said fractionating tower connecting with a gasoline quencher via a tube, and said quencher connecting to a condenser, said condenser linking to a separator of oil and water, a filter and a gasoline treating column in series, said gasoline treating column being connected with a filter and a product tank in series; the middle part of said fractionating tower connecting with a condenser, a separator of oil and water, a filter and a kerosene treating column in series, and said kerosene treating column connecting a filter and a product tank in series; and the low part of said fractionating tower connecting with a condenser, a separator of oil and water, a filter, and a diesel oil treating column in series, said diesel oil treating column connecting to a filter and a product tank in series; the bottom of said fractionating tower connecting to the cracker via a tube.
